(19) United States
(12) Patent Application Publication
(10) Pub. No.: US 2014/0066519 A1
(43) Pub. Date: Mar. 6, 2014

(54) USE OF XANTHOHUMOL AND/OR ISOXANTHOHUMOL AS AN AGENT FOR PREVENTING AND/OR COMBATING LIVER DISEASES

(71) Applicant: FLAXAN GMBH & CO. KG, Nurnberg (DE)

(72) Inventor: Claus HELLERBRAND, Regensburg (DE)

(73) Assignee: FLAXAN GMBH & CO. KG, Nurnberg (DE)

(21) Appl. No.: 14/074,332

(22) Filed: Nov. 7, 2013

Related U.S. Application Data
(63) Continuation-in-part of application No. 12/520,756, filed on Sep. 21, 2009.

(30) Foreign Application Priority Data
Dec. 22, 2006 (DE) 10 2006 062 264.2

(51) Int. Cl. A61K 31/12

(52) U.S. Cl. CPC A61K 31/12
USPC 514/689

(57) ABSTRACT

Method for the prevention and/or control of liver diseases, such as non-alcoholic steatohepatitis (NASH) even when eating a high fat diet. Prevention of NASH can be accomplished by preventing the liver from becoming a fatty liver even when eating a high fat diet by providing xanthohumol as a supplement to the high fat diet.

### PRIORITY INFORMATION

This application is a Continuation In Part of U.S. patent application Ser. No. 12/520,756 filed on Jun. 22, 2009, titled “USE OF XANTHOHUMOL AND/OR ISOXANTHOHUMOL AS AN AGENT FOR PREVENTING AND/OR COMBATING LIVER DISEASES,” which is herein incorporated by reference in its entirety.

### FIELD

Method for the prevention and/or control of liver diseases, such as non-alcoholic steatohepatitis (NASH).

### BACKGROUND

Xanthohumol is a prenylflavonoid which occurs in hops. Various studies have demonstrated the biological effects of xanthohumol.

For example, the anticarcinogenic effect of xanthohumol is described in EP 1 543 834 A1. It is known from EP 0 679 393 B1 that xanthohumol has a strong inhibitory effect on bone absorption, and therefore may be used as an agent for the treatment of osteoporosis.

103 08 864 A1 describes a novel brewing method for producing a beer which due to a special brewing process contains an elevated concentration of xanthohumol and therefore has increased health-promoting effects.

### BRIEF SUMMARY

An embodiment of the invention is a method for preventing and/or treating Non-Alcoholic Steatohepatitis (NASH) of a mammal eating a high fat diet comprises administering a xanthohumol having the formula

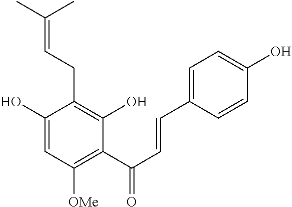 [SMILES: COc1cc(O)c(CC=C(C)C)c(O)c1C(=O)/C=C/c1ccc(O)cc1]

to liver cells, wherein the xanthohumol reduces alanine aminotransferase (ALT) secreted from and/or in the liver cells. In another embodiment, the xanthohumol inhibits messenger ribonucleic acid (mRNA) expression for proinflammatory cytokine tumor necrosis factor (TNF) by the liver cells. In another embodiment, the xanthohumol inhibits mRNA expression for Collagen Type I (Coll-I) by the liver cells. In another embodiment, the xanthohumol inhibits mRNA expression for profibrogenic factor tissue inhibitor of metalloproteinases 1 (TIMP-1) by the liver cells. In another embodiment, the xanthohumol inhibits fat accumulation by the liver cells. In an embodiment, the liver cells are mouse liver cells. The term “liver cell(s)” is used herein to include, but are not limited to, one or more or any combinations of, hepatodcytes (which when can produce ALT), proinflammatory macrophages (which produces TNF), hepatic stellate cells (which produces collegen), etc.

Another embodiment of the method for preventing and/or treating NASH of a liver of a mammal eating a high fat diet food comprises administering a xanthohumol having the formula

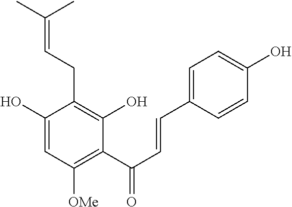 [SMILES: COc1cc(O)c(CC=C(C)C)c(O)c1C(=O)/C=C/c1ccc(O)cc1]

to the mammal, wherein the xanthohumol reduces ALT level in mammal's blood. The xanthohumol reduces and/or inhibits one or more of the following: ALT level in the liver, mRNA expression for proinflammatory cytokine TNF, mRNA expression for Coll-I, mRNA expression for profibrogenic factor TIMP-1 in the liver. The xanthohumol can also inhibit fat accumulation by the liver due to the high fat diet food. In an embodiment of the method, 50-500 mg of the xanthohumol is administered per kg body weight (kgBW) of the mammal.

### BRIEF DESCRIPTION OF THE DRAWINGS

FIG. 1 shows examples of therapeutic objectives for the use of xanthohumol.

FIG. 2 shows a schematic illustration of the efficacy of xanthohumol in preventing liver damage induced by viral infection and virus replication, respectively.

FIG. 3 shows the selective efficacy for the use of xanthohumol.

FIG. 4 shows a graphical illustration of the efficacy of the use of xanthohumol.

FIG. 5 shows a comparison of the growth of liver cancer cells (HepG2) over time as a function of the dosage of xanthohumol.

FIGS. 6 and 7 illustrate the effect of addition of xanthohumol for the prevention of transformation of the body's own physiologically “quiescent” (non-activated) hepatic stellate cells to activated hepatic stellate cells.

FIG. 8 shows an illustration of the effect of increasing dosages of xanthohumol on the activated hepatic stellate cells already present.

FIG. 9 shows the influence of the dosage of xanthohumol on the growth of the activated hepatic stellate cells.

FIG. 10 shows a comparison of the lifetime (proliferation) of liver cancer cells after administration of xanthohumol.

FIG. 11 shows how a progression of liver disease when eating a high fat diet.

FIG. 12 shows how the progression of liver disease can be prevented even when eating a high fat diet.

FIGS. 13-18 show graphs of experimental data of the effectiveness of xanthohumol when added as a supplement to a high fat diet.

### DETAILED DESCRIPTION

The present invention may be further understood with reference to the following description and the appended drawings, wherein like elements are referred to with the same reference numerals. The methods disclosed herein are directed towards health-promoting applications for xanthohumol and/or isoxanthohumol.

This object is achieved by the use of xanthohumol as an active substance for producing a preparation for the prevention and/or control of liver diseases.

The above object is further achieved by the use of isoxanthohumol as an active substance for producing a preparation for the prevention and/or control of liver diseases.

The claimed use has the advantage that by use of a natural active substance, liver diseases may be prevented and, through treatment, effectively eliminated or controlled. Xanthohumol or isoxanthohumol have no side effects. This allows effective prophylactic protection from chronic liver diseases over a long time period, in particular when taken regularly.

Xanthohumol and isoxanthohumol are particularly well suited for prevention or treatment of fatty liver disease and resulting hepatic inflammation and fibrosis. Surprisingly, studies have shown that xanthohumol inhibits metabolic mechanisms which are very important for liver damage mediated by obesity (e.g., overweight) and diabetes. Obesity and diabetes are responsible for the majority of cases of chronic liver disease and cirrhosis, and the trend is increasing. Collectively, chronic liver diseases have come to represent a significant economic problem. By continuous administration of xanthohumol or isoxanthohumol it is possible to provide effective prophylactic protection, without side effects, for the entire population.

Studies have further shown that xanthohumol or isoxanthohumol also have antiviral properties, and exhibit very good activity against hepatitis, in particular hepatitis B and hepatitis C. Hepatitis B or hepatitis C is the most common causative factor in chronic liver disease. Epidemiological studies in Germany have shown that approximately 2% of the population is infected with chronic hepatitis B or hepatitis C. This is also a problem of key social significance. By prophylactic administration of xanthohumol or isoxanthohumol it is possible on the one hand to effectively reduce the number of hepatitis cases, i.e., cases of hepatitis B and C, and on the other hand to favorably influence the course of an existing case of hepatitis.

There are currently no proven therapeutic administration forms for the treatment of hepatic fibrosis. Fibrosis can be inhibited or halted only by elimination of the harmful root cause, i.e., in the case of a hepatitis virus infection, for example, by elimination of the hepatitis virus. However, elimination of the root cause is successful in only a percentage of patients with chronic liver disease, and as a rule is not possible for patients with genetic liver disease. In the case of hepatitis virus infections, it has been necessary thus far to use medicaments having strong side effects. Even when such medicaments are used, elimination of the virus is achieved in only a percentage of patients. Fortunately, the use of xanthohumol or isoxanthohumol may provide a remedy.

Lastly, xanthohumol and isoxanthohumol also have anticarcinogenic effects. For liver cancer or hepatocelluar carcinoma (HCC), besides surgery there is currently no proven therapy which would improve the survival rate of patients. At the present time, surgical removal is successful only in a very small percentage of HCC patients, since by the time that diagnosis is made the HCC has usually become too large or has formed metastases. It has been shown that xanthohumol may be used in the treatment of liver cancer.

Furthermore, xanthohumol or isoxanthohumol may be used as a preventative specifically in persons with a high risk profile (genetic risk, persons with obesity, diabetics). With regard to administration, the invention provides for use by supplying xanthohumol or isoxanthohumol as an active ingredient of a pharmaceutical composition together with a pharmaceutically acceptable carrier such as mannite, sucrose, lactose, glucose, fructose, maltose, etc.

Xanthohumol or isoxanthohumol is particularly suitable when added as an active substance to a food, or mixed with a beverage.

According to one practical embodiment of the use according to the invention, xanthohumol or isoxanthohumol as an active substance is added in particular to reduce or suppress the activity of free oxygen radicals specifically in the liver. It has been found that when liver damage is present in any form, i.e., as the result of inflammation (for example, from viruses, excessive alcohol consumption, obesity, and/or diabetes or radiation exposure), free oxygen radicals are formed which may play a key role in the development of liver inflammation, hepatic fibrosis or cirrhosis of the liver, and liver cancer. Xanthohumol or isoxanthohumol inhibits the formation of free oxygen radicals or interferes with their activity. This results in the advantage that all three of the above-referenced damage mechanisms for the liver may be effectively influenced in equal measure by xanthohumol or isoxanthohumol.

In particular, it has been found that adding xanthohumol or isoxanthohumol is particularly suited for crucially influencing the NF-kappa B factor, i.e., in particular for reducing or suppressing the activity of the NF-kappa B factor. NF-kappa B is a signal mediator in the cell, and participates in the modulation of numerous cell functions. It has been found that the NF-kappa B factor plays a major role specifically in the three above-referenced damage mechanisms for the liver. NF-kappa B also plays an important role in the development and progression of NASH.

Furthermore, it has surprisingly been found that xanthohumol or isoxanthohumol may be administered in comparatively high dosages. According to the findings, no harmful effect from xanthohumol or isoxanthohumol occurs in any of the cells, even at high dosages, thus resulting in selective activity.

It has been found that with increasing dosages of xanthohumol or isoxanthohumol, for example beginning at a lower limit of 5 μM, a continuous increase in the positive effect can be observed, in particular up to a maximum limit of 100 μM. This results in the advantage that, depending on the intended use of the treatment agent (food with a proportion of xanthohumol or isoxanthohumol for daily intake as a preventative, or as a medicament for treatment), preparations having different dosages may be marketed for specific purposes.

For example, in a chronic infection several activity mechanisms may be present at the same time, so that the preparation according to the invention may be used to appropriately control different mechanisms of liver disease, e.g., liver inflammation, hepatic fibrosis and development of cirrhosis of the liver, and development of liver cancer as well as progression of liver cancer, all at the same time.

It is practical to use the active substance, i.e., the xanthohumol or isoxanthohumol or a metabolite thereof or a precursor thereof, in an administration form (application and/or dosage) which results in active substance concentrations of, for example, ≧0.01 μM, ≧1 μM, ≧5 μM, in particular ≧10 μM, in particular ≧20 μM, in particular ≧30 μM, in particular ≧40 μM, in particular ≧50 μM, in the liver. The active substance is preferably used in an administration form which results in a maximum active substance concentration of 100 μM in the liver.

Depending on the application, the particular active substance should be used in an administration form in such a way that the following ranges of active substance concentrations result in the liver: 1 to 100 μM, preferably 1-25 μM, preferably 1-10 μM, or 5-100 μM, preferably 10-50 μM, preferably 10-25 μM. The applicable ranges may be selected depending on the application. In particular, comparatively low doses are sufficient for the treatment of fibrosis, whereas increased doses are practical for treatment of liver cancer.

When xanthohumol or isoxanthohumol is administered in food or as a tablet, for example, due to absorption by the intestine relatively high xanthohumol or isoxanthohumol levels may result, but these are rapidly diluted after passage through the liver; i.e., no other organ has anywhere near such a high xanthohumol level.

With regard to recovery of xanthohumol from hops plants, reference is made to the entire disclosures of EP 0 679 393 B1 and EP 1 543 834 A1.

Instead of xanthohumol or isoxanthohumol, according to the present invention a metabolite thereof may also be used, in particular a metabolite which is produced in the liver by the P450 enzyme complex. Such metabolites are primarily xanthohumol glucoronides or sulfates, and methylated forms of xanthohumol or naringenins, in particular 8-prenylnaringenin. Naringenin, in particular 8-prenylnaringenin, is the final metabolite of xanthohumol. Likewise, instead of xanthohumol or isoxanthohumol a precursor thereof may be used which regenerates to form xanthohumol under chemical and/or physiological conditions.

All of the uses of xanthohumol and isoxanthohumol described in the present patent application for the treatment of liver diseases therefore also apply for the above-described metabolites and precursors.

According to the present invention, as active substance a composition may be used in which the xanthohumol and/or isoxanthohumol are present not in the pure form, but rather in the form of a hops extraction product. It has been found that in addition to xanthohumol or isoxanthohumol, carrier constituents are present as the result of the production process which are able to further assist in absorption of the active substance into the organism and thereby help boost efficacy.

The dosage for administration of the active substance relative to the respective (pure) fraction of active substance is advantageously greater than 0.01 mg/kg body weight/day, preferably greater than 0.1 mg/kg body weight/day, preferably greater than 1 mg/kg body weight/day, preferably greater than 10 mg/kg body weight/day, preferably greater than 50 mg/kg body weight/day, preferably greater than 100 mg/kg body weight/day, whereby the body weight refers to the body weight of a person.

The dosage for administration relative to the respective (pure) fraction of active substance is advantageously less than 161 mg/kg body weight/day, preferably less than 50 mg/kg body weight/day, preferably less than 10 mg/kg body weight/day, preferably less than 1 mg/kg body weight/day, preferably less than 0.1 mg/kg body weight/day, whereby the body weight refers to the body weight of a person.

The dosage for administration relative to the respective (pure) fraction of active substance is advantageously in a range of 0.01 to 161 mg/kg body weight/day, preferably 0.05 to 120 mg/kg body weight/day, preferably 0.1 to 100 mg/kg body weight/day, preferably 0.5 to 80 mg/kg body weight/day, preferably 1 to 80 mg/kg body weight/day, preferably 5 to 80 mg/kg body weight/day, preferably 10 to 80 mg/kg body weight/day, whereby the body weight refers to the body weight of a person.

The proportions of xanthohumol or isoxanthohumol advantageously are in a range of 0.1 wt-%-99 wt-%, preferably 5 wt-%-99 wt-%, preferably 10 wt-%-99 wt-%, preferably 20 wt-%-99 wt-%, preferably 30 wt-%-99 wt-%, preferably 40 wt-%-99 wt-%, preferably 50 wt-%-99 wt-%, preferably 60 wt-%-99 wt-%, preferably 70 wt-%-99 wt-%.

If further constituents, in particular natural constituents resulting from the recovery of xanthohumol from hops, are present in addition to the xanthohumol as active substance, this may even increase the efficacy, since these constituents result in improved absorption of the active substance in the organism.

Alternatively, the xanthohumol or isoxanthohumol may also be used in pure form. In addition, according to the present invention it is also possible to use xanthohumol or isoxanthohumol in synthesized form.

According to a further embodiment, the xanthohumol, isoxanthohumol, a metabolite thereof, and/or a precursor thereof are used in combination with at least one additional active substance. This active substance may preferably be one which positively influences the tolerability and/or absorption in the body, and/or the efficacy and/or stability and/or handling characteristics, of the active substance to be administered.

The xanthohumol, isoxanthohumol, a metabolite thereof, and/or a precursor thereof may be used in combination with or on the basis of a salt, in particular an alkali or alkaline earth salt. The agent to be administered may in particular be used in the form of a liquid, suspension, or emulsion, in the form of nanoparticles, or as a powder or gel. The administration may be carried out as an independent medicament, or also as an additive to a liquid or solid food, depending on whether therapy or prophylaxis is desired.

The active substance may be administered using solvents, carrier substances, or additives such as starches, dextrin, in particular cyclodextrin or maltodextrin, proteins, methyl cellulose, carbomethoxycellulose, or xanthan gum which are suitable for pharmaceuticals, nutrients, or foods.

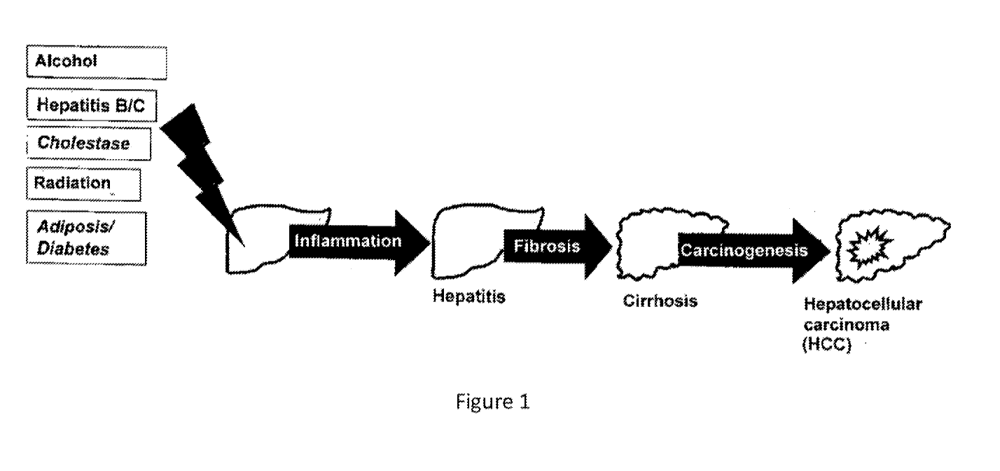

The studies according to the following FIGS. 1-9 were carried out using xanthohumol in pure form (>98%).

### EXAMPLE 1

A composition of a medicament is provided below as an example.

Powdered mixture for direct pressing

| | | |
|---|---|---|
| Xanthohumol (pure substance) | 5 | g |
| Microcrystalline cellulose | 10 | wt-% |
| Sodium carboxymethyl starch | 3 | wt-% |
| Highly dispersed silica | 1 | wt-% |
| Magnesium stearate | 1 | wt-% |
| Tablettose (lactose monohydrate) | to make 100 | wt-% |

### EXAMPLE 2

A composition of a food with added xanthohumol as active substance is provided below as an example.
Xanthohumol (pure substance in powdered form) 500 mg per 200 mL milk product (creamy, for example yogurt)

Due to the ease of admixture into a creamy food, the above composition for a food allows optimal administration of the required quantity of xanthohumol.

FIG. 1 shows the therapeutic objectives for the use of xanthohumol. The illustration represents the chain of activity mechanisms, starting from a liver disease resulting from alcohol, viruses, radiation, adiposis/obesity, and/or diabetes, for example, all the way to liver cancer. The use of a preparation containing xanthohumol and/or isoxanthohumol advantageously interferes with all stages of the activity chain according to FIG. 1. However, xanthohumol or isoxanthohumol may also be used successfully in a targeted manner in the treatment of individual stages of the activity sites.

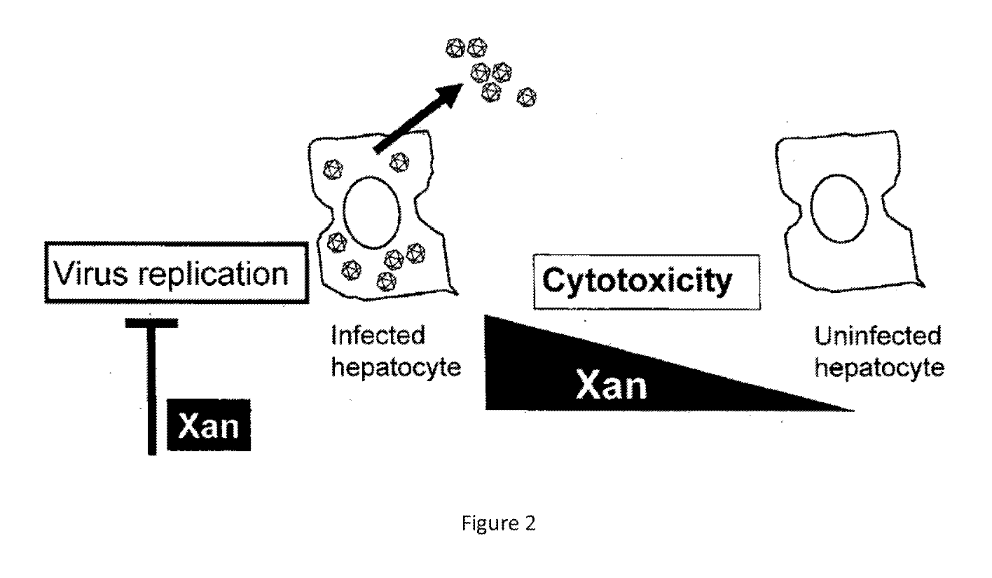

FIG. 2 shows a schematic illustration of the efficacy of xanthohumol and/or isoxanthohumol for viral damage to the liver, in particular as the result of hepatitis B and C. It has been found that the above-referenced active substances advantageously not only inhibit replication of the virus, but also ensure selective destruction of the body's own liver cells already affected by the virus while leaving healthy liver cells undamaged. Thus, use of the invention allows a targeted therapy for reduction or elimination of liver cells infected with the virus.

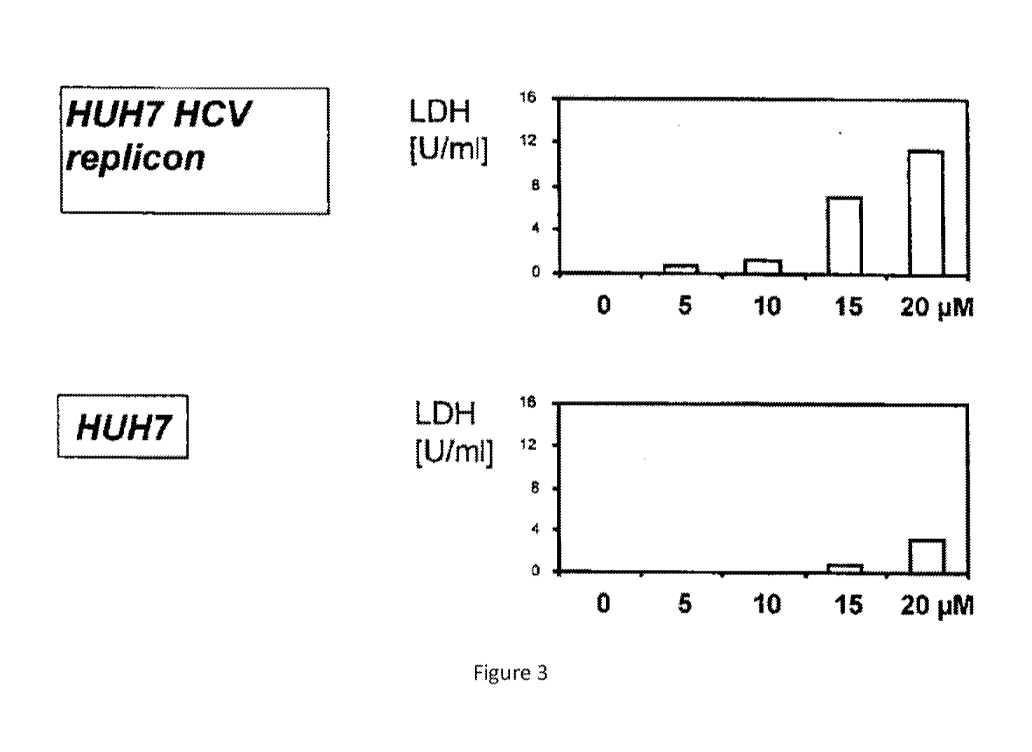

On the basis of comparative diagrams, FIG. 3 shows the selective efficacy for the use of xanthohumol or isoxanthohumol for liver cells infected with hepatitis C (HUH7 HCV replicon), compared to liver cells not infected with hepatitis C (HUH7).

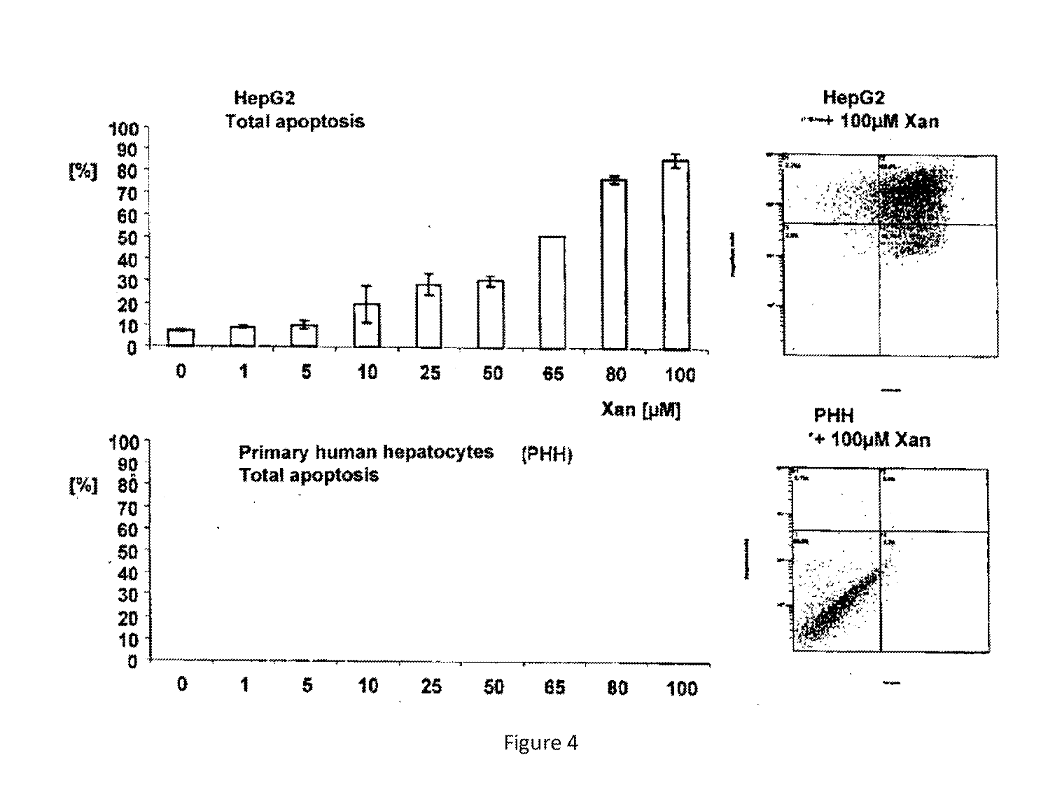

FIG. 4 shows a graphical illustration of the efficacy of the use of xanthohumol with regard to apoptosis (programmed cell death) of liver cancer cells (HepG2) compared to healthy liver cells (primary human hepatocytes).

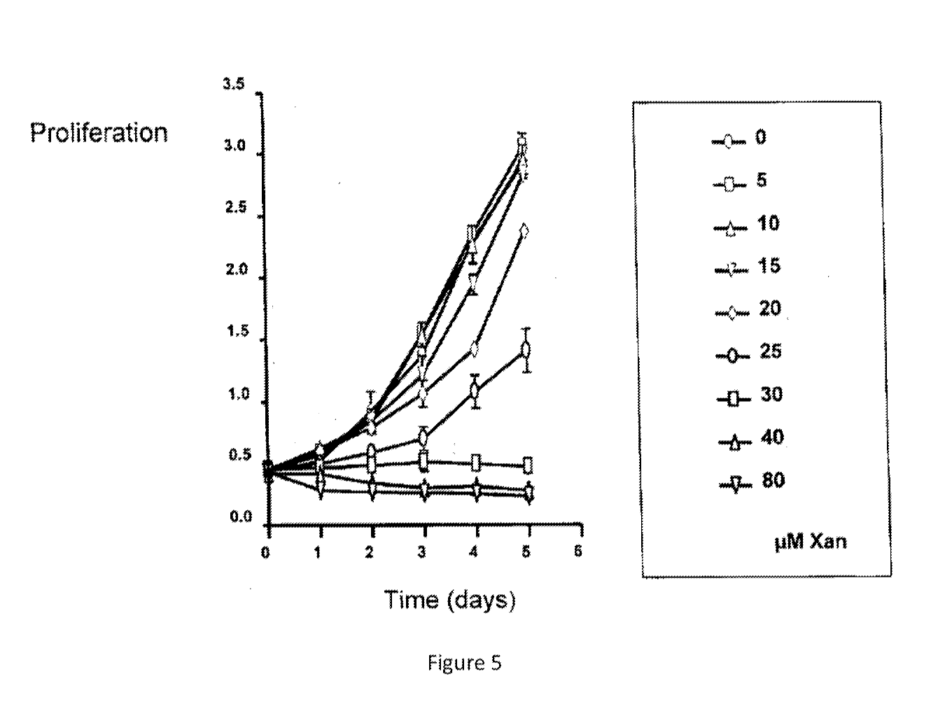

FIG. 5 shows a comparison of the growth of liver cancer cells (HepG2) over time as a function of the dosage of xanthohumol. As clearly shown in the illustration, the growth of the cancer cells is progressively inhibited with increasing concentrations of xanthohumol.

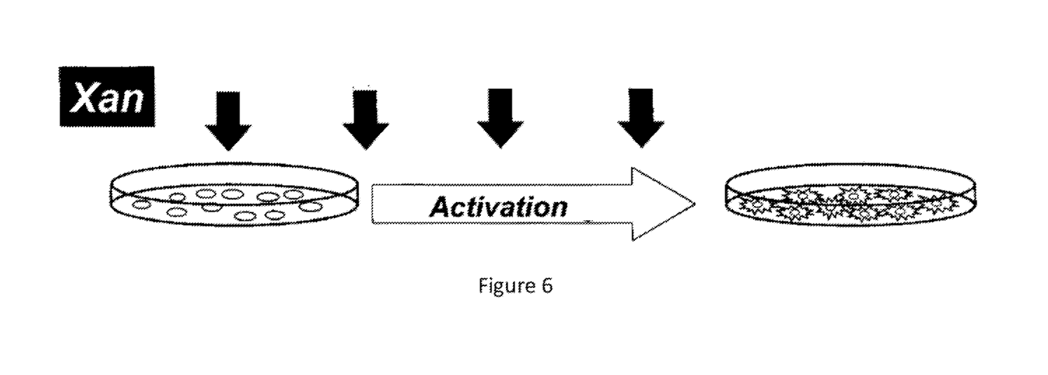

FIGS. 6 and 7 illustrate the effect of addition of xanthohumol for the prevention of transformation of the body's own, physiologically “quiescent” (non-activated) hepatic stellate cells to activated hepatic stellate cells, which are responsible for scarring of the liver in cirrhosis of the liver.

As shown in FIG. 7, the formation of scar tissue is increasingly suppressed as the dosage of xanthohumol increases.

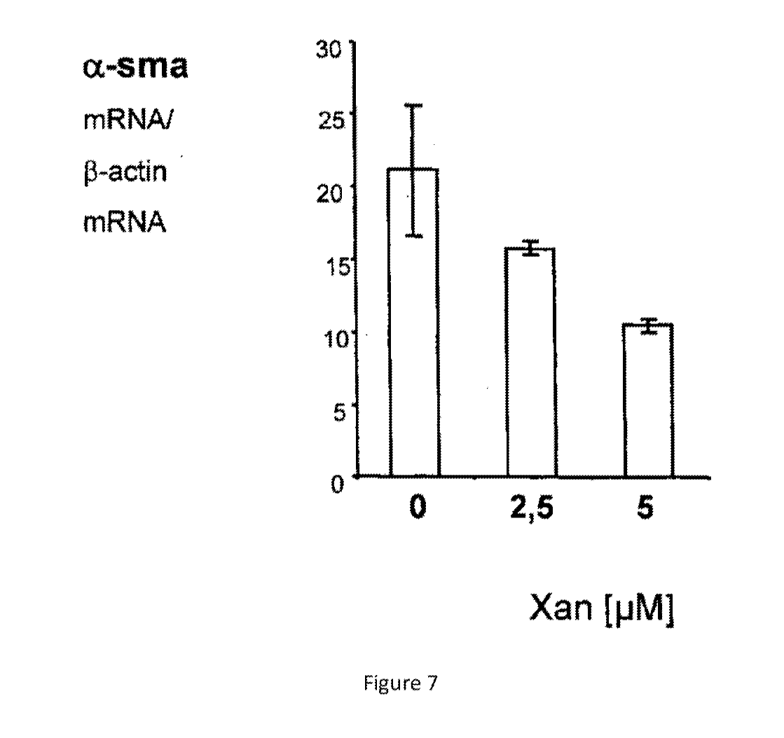

FIG. 8 shows an illustration of the effect of increasing dosages of xanthohumol on the activated hepatic stellate cells already present. From the illustration according to FIG. 8 it is seen that an increased effect of destruction (LDH) of activated hepatic stellate cells results from an increasing dosage of xanthohumol.

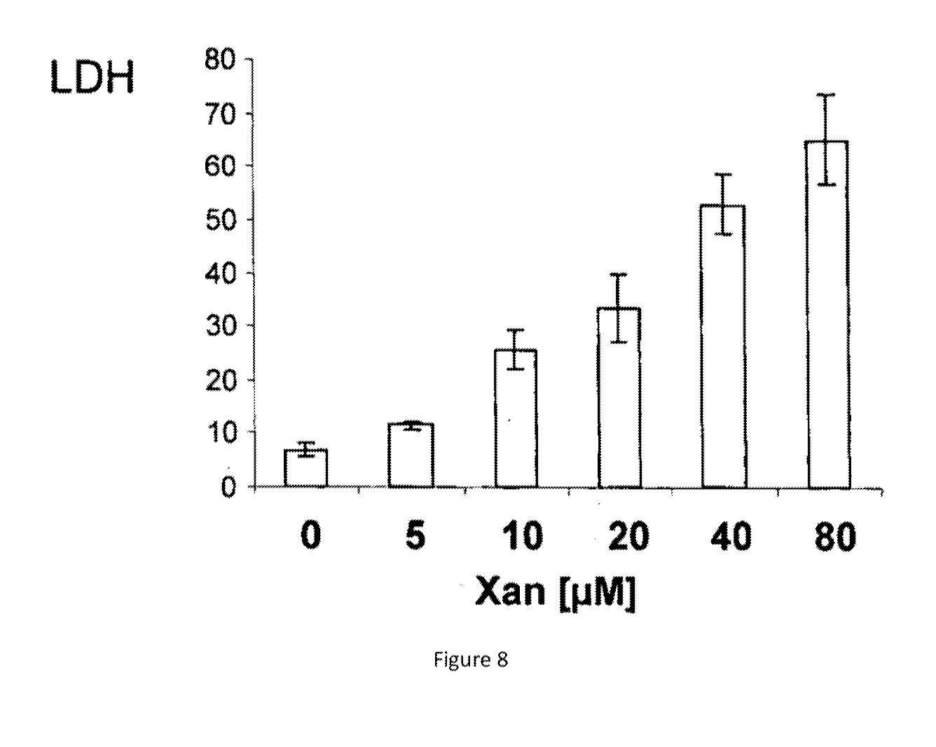

FIG. 9 shows the influence of the dosage of xanthohumol on the growth of the activated hepatic stellate cells.

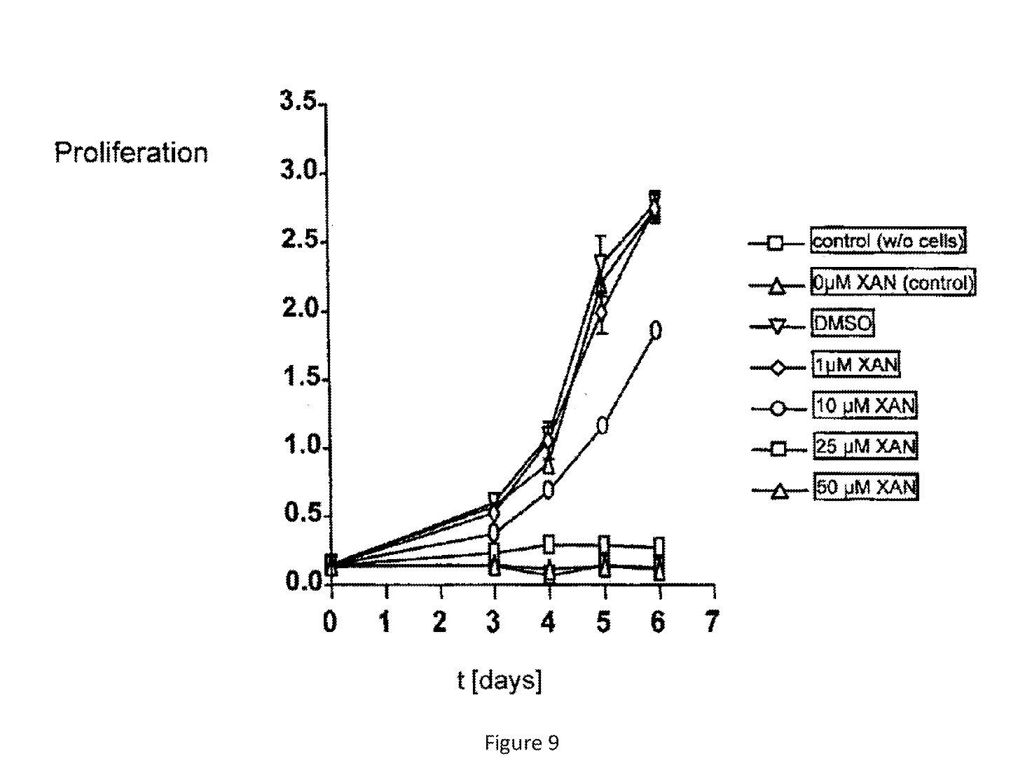

FIG. 10 shows a comparison of the lifetime (proliferation) of liver cancer cells after administration of xanthohumol in pure form (>98%) or in a form in which xanthohumol is present in a proportion of 60%. The latter case represents the xanthohumol recovered from hops extract in a conventional commercial process, containing additional natural constituents. In the figure, the lower the bars, the more cells that experience inhibition of growth.

It is demonstrated that use of 60% xanthohumol results in an even stronger effect than from xanthohumol in pure form. This is attributed to the fact that for the natural xanthohumol, the remaining constituents have a carrier function and therefore supply the active substance to the organism in a more effective manner.

Further, the present invention relates to the use of xanthohumol as an active substance for the prevention and/or control of NASH. NASH is a “silent” liver disease which can occur to people who consume little or no alcohol. NASH is known as the “silent” liver disease because most people with NASH are not aware that they have it. Regardless, NASH can lead to permanent damage to the liver. In the U.S., it has been estimated that NASH affects 2 to 5 percent of the population, while 10 to 20 percent of the population have a condition known commonly as “fatty liver” (wherein there is fat in their liver, but no inflammation or liver damage). Diagnosed “fatty liver” condition is known as nonalcoholic fatty liver disease (NAFLD). Number of people suffering from NASH and/or NAFLD is rising. It is theorized that the number of people suffering from NASH and/or NAFLD is rising because the number of people, including children, suffering from obesity is rising. There appears to be a link between obesity and NASH and/or NAFLD. Further, there appears that high fat diet can lead to obesity.

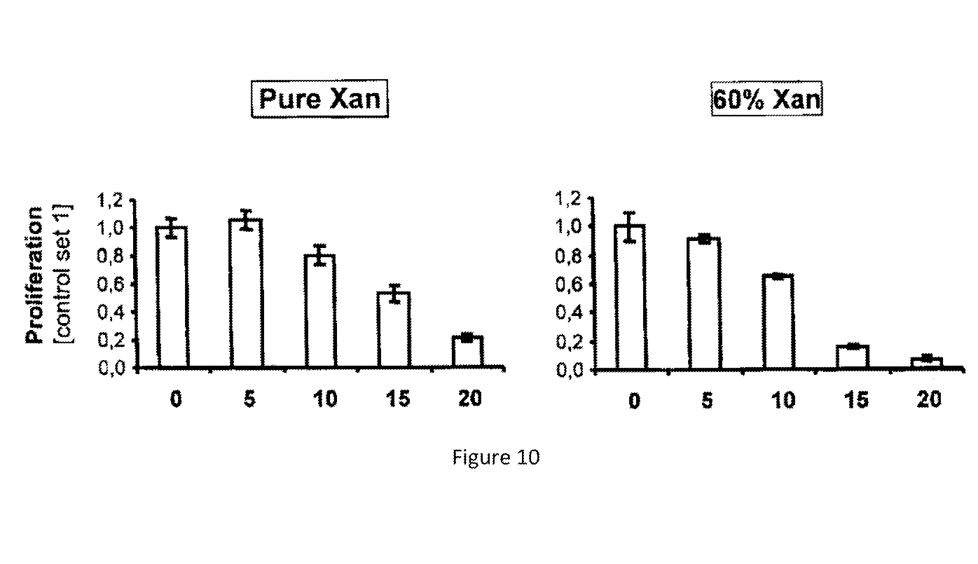

FIG. 11 shows a flow chart 100 of how NASH 102 can develop in a liver from eating a high fat diet 104. Eating a high fat diet (HFD) 104 can lead to fatty liver 106. Fatty liver 106 can lead to inflammation 108 of the liver. Inflamed liver 108 can lead to fibrosis 110 of the liver. The combination of these three disease mechanisms, fatty liver 106, inflammation 108, and liver fibrosis 110 defines NASH 102.

In physiological conditions, fat accumulated by liver cells do not remain in the liver cells. The fat (e.g., lipids) can be metabolized and/or secreted by the liver cells. Accordingly, fat accumulation is a dynamic process. Progression from fatty liver 106 to NASH 102 is a dynamic process. Xanthohumol can be provided as an effective treatment that can reduce further progression of liver diseases such as fatty liver 106, inflammation of the liver 108, fibrosis of the liver 110, NASH 102, and/or preventions therefore by inhibiting progression of liver damage.

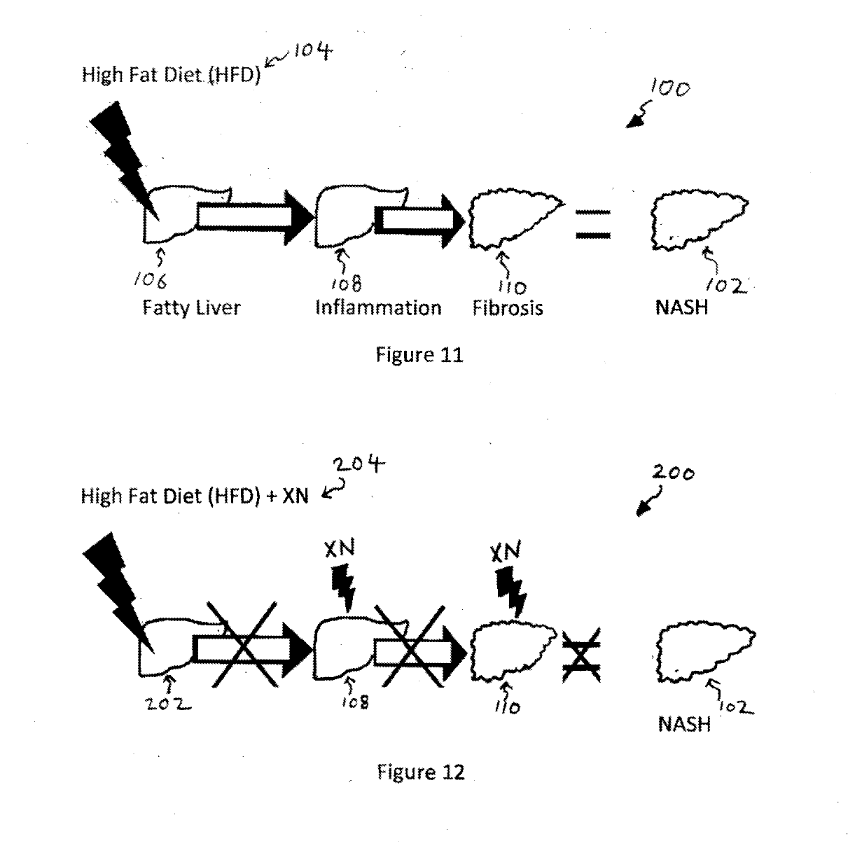

An effective method of preventing NASH 102 can be performed by preventing the liver from becoming a fatty liver 106 even when eating a high fat diet. FIG. 12 shows a flow chart 200 of how this prevention can be accomplished by inhibiting the liver 202 from accumulating fat. Such inhibition can be accomplished by providing xanthohumol (XN) when eating a high fat diet (HFD) 204. Xanthohumol, under physiological conditions, can inhibit liver cells and/or the liver 202 from fat accumulation. Providing xanthohumol to a liver (or liver cells) when eating a high fat diet prevents the liver 202 from developing into fatty liver. Such prevention can stop the progression of the liver 202 from becoming inflamed and developing fibrosis. Thus, xanthohumol can prevent the combination of the three disease mechanisms, fatty liver 106, inflammation 108, and liver fibrosis 110 which defines NASH 102. Therefore, xanthohumol can prevent and/or treat NASH 102. That is, because xanthohumol prevents the fatty liver state even when eating a high fat diet, the liver 202 does not progress (or slows the progression) to inflammation 108 and fibrosis 110 caused by eating high fat diet. Thus, NASH 102 can be prevented by providing xanthohumol when eating a high fat diet 204.

Another effective method of preventing NASH 102 can be performed by inhibiting inflammation 108 of the liver even when eating a high fat diet. Another effective method of preventing NASH 102 can be performed by inhibiting fibrosis 110 of the liver even when eating a high fat diet. Even more effective method of preventing NASH 102 can be performed by inhibiting fat accumulation, inflammation, and fibrosis. Inhibition of fat accumulation, inflammation, and/or fibrosis can also be an effective treatment for slowing down or preventing further progression of the liver disease state (including NASH). It has been found that xanthohumol prevents and/or inhibits further progression of NASH 102 by inhibiting fat accumulation, inflammation 108, and fibrosis 110. It has been found that xanthohumol directly inhibits inflammation 108 caused by steatosis (fat accumulation or lipid accumulation) of liver cells and that xanthohumol also directly inhibits the reception of fatty acids into liver cells (i.e., the formation of steatosis). Inhibition of the reception of fatty acids into liver cells was determined in vitro tests and in vivo experiments. According to in vitro tests, the results have shown that an increased amount of xanthohumol lessens fatty degeneration, yet xanthohumol does not affect the viability of the liver cells.

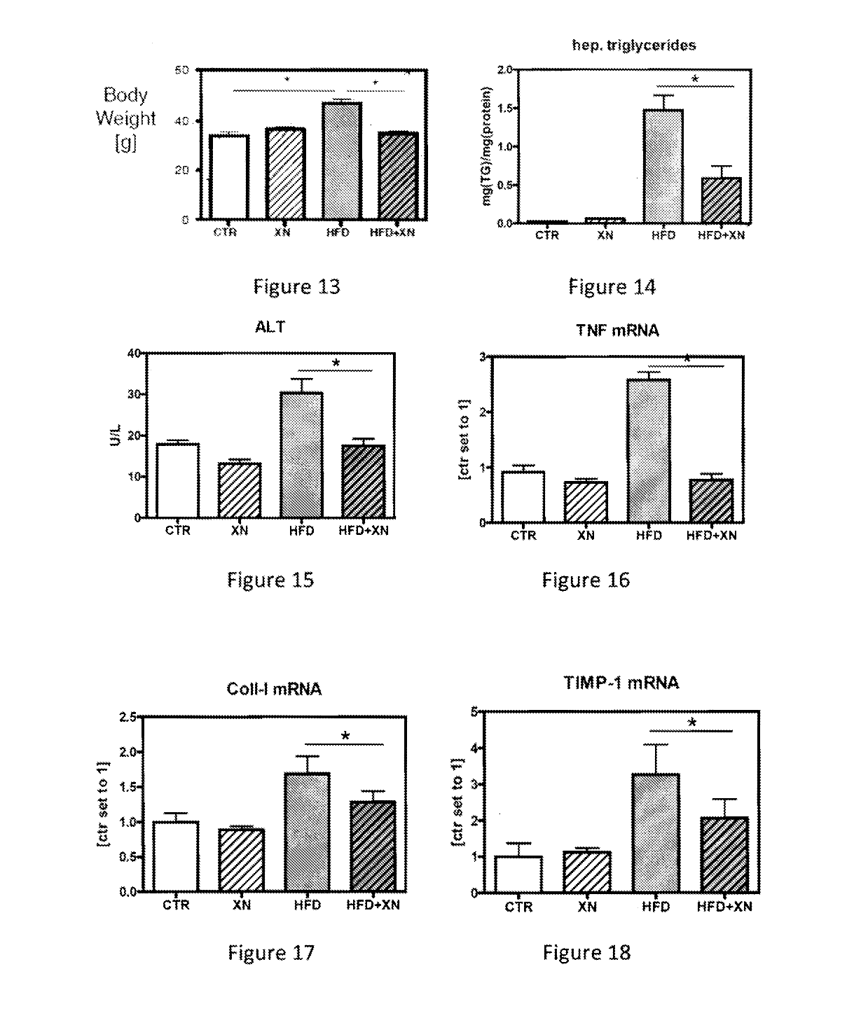

In vivo studies correlate with the in vitro studies. FIGS. 13-18 show comparative graphs of in vivo experimental data showing the effectiveness of xanthohumol when eating a high fat diet. The experiments involved using male C57BL/6N mice purchased from Charles River Laboratories (Sulzfeld, Germany). After two weeks of acclimatization, the mice were fed either standard diet (CTR), standard diet with xanthohumol (XN), high fat diet without providing xanthohumol (HFD), or high fat diet and xanthohumol (HFD+XN). High fat diet in the experiments included a NASH-inducing diet which includes sucrose, cholesterol, and fats rich in saturated fatty acids. In detail, the high fat diet included pork lard (15%), beef tallow (15%), palmitic acid (4%), stearic acid (4%), cholesterol (0.2%), and sucrose (30%). The feeding lasted for 20 weeks. Each experimental group had six mice. The XN and HFD+XN mice were provided with 500 mg of xanthohumol per kg of body weight (kgBW).

FIG. 13 shows a chart of the weight gain of the mice from the experiments (*: p<0.05). The mice that ate high fat diet (HFD) gained weight the most. The mice that ate high fat diet and also provided with xanthohumol (HFD+XN) did not have the weight gain of the HFD mice. In fact, the weight gain by the HFD+XN mice was similar to the CTR mice that did not eat the high fat diet. Accordingly, FIG. 13 shows that xanthohumol can substantially inhibit weight gain while eating a high fat diet.

FIG. 14 shows a chart of the detected measurement of hepatic triglycerides (TG) of the mice from the experiments (*: p<0.05). The mice that ate high fat diet (HFD) had the highest level of hepatic TG (mg)/protein ratio (mg). The mice that ate high fat diet and also provided with xanthohumol (HFD+XN) had a substantial reduction of level of hepatic TG (mg)/protein ratio (mg) as compared to the HFD mice. Accordingly, FIG. 14 shows that xanthohumol can reduce hepatic TG (i.e., ratio of mg(TG)/mg(protein)) while eating a high fat diet substantially (e.g., by more than 50% compared to eating high fat diet without xanthohumol). The lowest detected levels of mg(TG)/mg(protein) were the control (CTR) mice and the mice provided with xanthohumol eating the same diet as the control mice (i.e., not eating high fat diet) (XN). Thus, xanthohumol can prevent fatty liver and/or treat further progression of fatty liver even while eating a high fat diet. Accordingly, a method for preventing and/or treating fatty liver for a mammal (e.g., mouse, human, etc.) eating a high fat diet includes providing xanthohumol to the mammal's liver, wherein the xanthohumol inhibits accumulation of fat in the liver which otherwise can lead to liver damage (e.g., fatty liver, inflammation, fibrosis, and/or NASH) due to high fat diet.

FIG. 15 shows an ALT test results. NASH can be diagnosed when a test shows elevations of such as ALT in blood. The ALT test generally measures an amount of the ALT enzyme in the blood. While ALT is found mainly in the liver, smaller amounts of ALT are found in other organs (e.g., kidneys, heart, muscles, and pancreas). Although low levels of ALT are normally present in blood, when the liver is damaged and/or diseased, the liver can release ALT into the bloodstream. Accordingly, ALT levels in the blood can be detected for diagnosing liver damage and/or disease. As shown in FIG. 15, ALT levels can be reduced by consuming xanthohumol when eating a high fat diet. FIG. 15 shows a chart of the detected measurement of ALT of the mice from the experiments (*: p<0.05). The mice that ate high fat diet (HFD) had the highest level of ALT (measured in units per liter, or U/L). The mice that ate high fat diet and also provided with xanthohumol (HFD+XN) had a substantial reduction of level of ALT as compared to the HFD mice. The reduction was by almost 50% of the HFD mice, such that the ALT of the HFD+XN mice had nearly the same ALT level as the control mice (CTR). Thus, xanthohumol can prevent NASH and/or treat further progression of NASH even while eating a high fat diet. The lowest detected levels of ALT were in the mice provided with xanthohumol eating the same diet as the control mice (i.e., not eating high fat diet) (XN). Accordingly, a method for reducing ALT in blood for a mammal (e.g., mouse, human, etc.) eating a high fat diet includes providing xanthohumol to the mammal's liver, wherein the xanthohumol inhibits accumulation of fat in the liver which otherwise can lead to liver damage (e.g., fatty liver, inflammation, fibrosis, and/or NASH) due to high fat diet. Thus, xanthohumol can prevent NASH and/or treat further progression of NASH even while eating a high fat diet.

FIG. 16 shows a chart of the detected measurement of the messenger ribonucleic acid (mRNA) expression of proinflammatory cytokine tumor necrosis factor (TNF) of the mice from the experiments (*: p<0.05; normalized so that CTR is set to 1). The TNF measurement is an indicator of inflammation in the liver. The mice that ate high fat diet (HFD) had the highest level of TNF mRNA. The mice that ate high fat diet and also provided with xanthohumol (HFD+XN) had a substantial reduction of level of TNF mRNA as compared to the HFD mice. The reduction was by more than 50% of the HFD mice, such that the TNF mRNA of the HFD+XN mice had nearly the same TNF mRNA level as the control mice (CTR). The lowest detected levels of TNF mRNA were in the mice provided with xanthohumol eating the same diet as the control mice (i.e., not eating high fat diet) (XN). Accordingly, a method for reducing inflammation in the liver (and/or mRNA expression of TNF) for a mammal (e.g., mouse, human, etc.) eating a high fat diet includes providing xanthohumol to the mammal's liver, wherein the xanthohumol inhibits accumulation of fat in the liver and mRNA expression of TNF, which otherwise can lead to liver damage (e.g., fatty liver, inflammation, fibrosis, and/or NASH) due to high fat diet. Thus, xanthohumol can prevent inflammation of the liver and/or NASH and/or treat further progression of inflammation of the liver and/or NASH even while eating a high fat diet.

FIGS. 17 and 18 show charts of the detected measurement of hepatic mRNA expressions of Collagen Type I (Coll-I) and profibrogenic factor tissue inhibitor of metalloproteinases 1 (TIMP-1), respectively, of the mice from the experiments (*: p<0.05; normalized so that CTR is set to 1). These methodologies are established markers detecting hepatic fibrosis (e.g., fibrosis of the liver). The mice that ate high fat diet (HFD) had the highest levels of Coll-I mRNA and TIMP-1 mRNA. The mice that ate high fat diet and also provided with xanthohumol (HFD+XN) had substantial reductions of Coll-I mRNA and TIMP-1 mRNA, compared to the HFD mice. The detected levels of Coll-I mRNA and TIMP-1 mRNA of the mice provided with xanthohumol eating the same diet as the control mice (i.e., not eating high fat diet) (XN) was about the same as the control mice (CTR). Accordingly, a method for reducing mRNA expression of Coll-I for a mammal (e.g., mouse, human, etc.) eating a high fat diet includes providing xanthohumol to the mammal, wherein the xanthohumol inhibits accumulation of fat in the liver which can lead to liver damage, such as NASH. Further, a method for reducing mRNA expression of TIMP-1 for a mammal (e.g., mouse, human, etc.) eating a high fat diet includes providing xanthohumol to the mammal, wherein the xanthohumol inhibits accumulation of fat in the liver which can lead to liver damage (e.g., fatty liver, inflammation, fibrosis, and/or NASH) due to high fat diet. Thus, xanthohumol can prevent hepatic fibrosis and/or NASH and/or treat further progression of hepatic fibrosis and/or NASH even while eating a high fat diet.

As shown in FIGS. 13-18, about 500 mg of xanthohumol per kgBW in a mouse can have beneficial effects in a mouse liver when the mouse is eating a high fat diet. It is generally understood that mouse liver is a model for other mammal livers, such as for example human livers. Accordingly, it would be expected that xanthohumol would have the same or similar properties for other mammalian livers, such as for example, human livers. Accordingly, it can be estimated that about 50 mg of xanthohumol per kgBW in a human can have beneficial effect in a human liver when the human is eating a high fat diet. It is estimated that 50-500 mg of xanthohumol per kgBW in a mammal can have even more beneficial effect in a liver when the mammal is eating a high fat diet. It is also estimated that 100-200 mg of xanthohumol per kgBW in a human can have even more beneficial effect in a human liver when the human is eating a high fat diet. Lower levels than the above may also have beneficial effects.

Preferred embodiments have been described. Those skilled in the art will appreciate that various modifications and substitutions are possible, without departing from the scope of the invention as claimed and disclosed, including the full scope of equivalents thereof.

What is claimed is:

1. A method for preventing and/or treating non-alcoholic steatohepatitis (NASH) of a mammal eating a high fat diet, comprising:
administering a xanthohumol having the formula 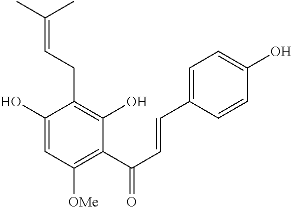 [SMILES: COc1cc(O)c(CC=C(C)C)c(O)c1C(=O)/C=C/c1ccc(O)cc1]
to liver cells, wherein the xanthohumol reduces alanine aminotransferase (ALT) in the liver cells.

2. The method according to claim 1, wherein the xanthohumol inhibits alanine aminotransferase (ALT) secreted from the liver cells.

3. The method according to claim 1, wherein the xanthohumol inhibits messenger ribonucleic acid (mRNA) expression for proinflammatory cytokine tumor necrosis factor (TNF) by the liver cells.

4. The method according to claim 1, wherein the xanthohumol inhibits messenger ribonucleic acid (mRNA) expression for Collagen Type I (Coll-I) by the liver cells.

5. The method according to claim 1, wherein the xanthohumol inhibits messenger ribonucleic acid (mRNA) expression for profibrogenic factor tissue inhibitor of metalloproteinases 1 (TIMP-1) by the liver cells.

6. The method according to claim 1, wherein the xanthohumol inhibits fat accumulation by the liver cells.

7. The method according to claim 1, wherein the liver cells are mouse liver cells.

8. A method for preventing and/or treating non-alcoholic steatohepatitis (NASH) of a liver of a mammal eating a high fat diet food, comprising:
administering a xanthohumol having the formula 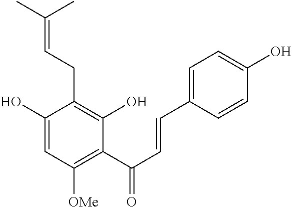 [SMILES: COc1cc(O)c(CC=C(C)C)c(O)c1C(=O)/C=C/c1ccc(O)cc1]
to the mammal, wherein the xanthohumol reduces alanine aminotransferase (ALT) level in mammal's blood.

9. The method according to claim 8, wherein the xanthohumol reduces alanine aminotransferase (ALT) level in the liver.

10. The method according to claim 8, wherein the xanthohumol inhibits messenger ribonucleic acid (mRNA) expression for proinflammatory cytokine tumor necrosis factor (TNF) in the liver.

11. The method according to claim 8, wherein the xanthohumol inhibits messenger ribonucleic acid (mRNA) expression for Collagen Type I (Coll-I) in the liver.

12. The method according to claim 8, wherein the xanthohumol inhibits messenger ribonucleic acid (mRNA) expression for profibrogenic factor tissue inhibitor of metalloproteinases 1 (TIMP-1) in the liver.

13. The method according to claim 8, wherein the xanthohumol inhibits fat accumulation by the liver due to the high fat diet food.

14. The method according to claim 8, wherein the mammal is a mouse.

15. The method according to claim 8, wherein 50-500 mg of the xanthohumol is administered per kg body weight (kgBW) of the mammal.

16. A method for preventing and/or treating non-alcoholic steatohepatitis (NASH) of a liver of a mammal eating a high fat diet food, comprising:
administering a xanthohumol having the formula 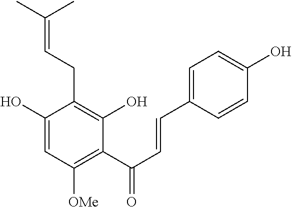 [SMILES: COc1cc(O)c(CC=C(C)C)c(O)c1C(=O)/C=C/c1ccc(O)cc1]
to the mammal, wherein the xanthohumol inhibits messenger ribonucleic acid (mRNA) expression for proinflammatory cytokine tumor necrosis factor (TNF) in the liver.

17. The method according to claim 16, wherein the xanthohumol inhibits messenger ribonucleic acid (mRNA) expression for Collagen Type I (Coll-I) in the liver.

18. The method according to claim 16, wherein the xanthohumol inhibits messenger ribonucleic acid (mRNA) expression for profibrogenic factor tissue inhibitor of metalloproteinases 1 (TIMP-1) in the liver.

19. The method according to claim 16, wherein the xanthohumol inhibits fat accumulation by the liver due to the high fat diet food.

20. The method according to claim 16, wherein 50-500 mg of the xanthohumol is administered per kg body weight (kgBW) of the mammal.
